SEQUENCE LISTING (5 sequences)

SEQ ID NO: 1 - DNA, 1075 bp, Homo sapiens
cctcctgcttctgtgtcaatgctccattcccaggctcctagtaggggcaaaagggcctgctgggtgcaggagaacctccagtcccaggtatggctgcagcccgtgccaatcctgttccagccacttgaggtgccgacagcattggagttggagggaaagttggggtctgtgtgtttgagctctgtcacatggacaatggggtagaggggagcctggccctttgaacagggctcaggaccaggagtgacttcctctccaggttgccagtcctgctccatccctcatgtcctggaagagagagagagacgaggtggctgcccgagtccacgtgaaaggcagcctctgacctcttgtccccaggcaggtggtggcggctggcaacacactcatagggccccatgagggttcaggattggagggggtagatttgggggcctcttagctctcaaagaggggcaatttaacgggcagaggtccatttggatccagaccattcacaggctcgaaggggacactgggagttggggctgggctcaggcctgctggggaaccaaaacttctgcttctgaggggtgtcagtaattccattttgactctgggggagccattttaaatctgtttatttccttctcatagaatcatgggtgagagctggcatggcccctagaggtcatttggggtccagctgcctcaccgtatcaatgaggaaactgaggcccagaaaagaaaagcatttttgcccagagtccctcagtgagtcctggttccgtacctgctttctgccagggaccaaactcccgttagtcattcctggtgtcagccaggcagaggagcaggtgggggagagggcgtgggaggagggaggtgaggcagctccctccgagtggaagctcacagtcagctgcagcgccgcccctgccctgactgcagctgactggcaggggcacctggaggcagcgggttcgggcgcagtatgagagggcttaccctctgcggcagtgggagacaggaccaggcagctccccacatccttccacatccgagctccaggggtctctggcttgtcctttcacag

SEQ ID NO: 2 - DNA, 23 bp, artificial
  primer
caggttgccagtcctgctccatc

SEQ ID NO: 3 - DNA, 22 bp, artificial
  primer
acctgcctggggacaagaggtc

SEQ ID NO: 4 - DNA, 25 bp, artificial
  primer
gttcgcagcgtcacccggatcttcg

SEQ ID NO: 5 - DNA, 23 bp, artificial
  primer
aggcttgcagaatgcggaacacc

(19) United States
(12) Patent Application Publication
(10) Pub. No.: US 2019/0142832 A1
(43) Pub. Date: May 16, 2019

(54) Marker for Cancer Prognosis and Methods Related Thereto

(71) Applicant: National Jewish Health, Denver, CO (US)

(72) Inventors: Aftab Ahmad, Hoover, AL (US); Carl W. White, Denver, CO (US)

(21) Appl. No.: 16/168,150

(22) Filed: Oct. 23, 2018

Related U.S. Application Data
(63) Continuation of application No. 14/818,557, filed on Aug. 5, 2015, now abandoned.
Application No. 14/818,557 is a continuation of application No. 13/662,898, filed on Oct. 29, 2012, now abandoned.
Application No. 13/662,898 is a continuation of application No. 13/087,607, filed on Apr. 15, 2011, now abandoned.
Application No. 13/087,607 is a continuation of application No. 12/271,051, filed on Nov. 14, 2008, now abandoned.
(60) Provisional application No. 60/987,892, filed on Nov. 14, 2007.

(51) Int. Cl. A61K 31/519; G01N 33/574; G01N 33/569; C12Q 1/6886; G01N 33/68; A61K 9/00

(52) U.S. Cl. CPC A61K 31/519; G01N 33/57407; G01N 33/56966; C12Q 1/6886; G01N 33/57492; C12Q 2600/106; A61K 9/007; C12Q 2600/178; G01N 2800/56; C12Q 2600/118; C12Q 2600/158; G01N 33/6893

(57) ABSTRACT

The present invention is related to the novel discovery that HIF-2α, but not HIF-1α, selectively regulates adenosine A_{2A} receptor in endothelial cells, thereby revealing a unique and hitherto unknown pathway by which HIF-2α can regulate angiogenesis independent of HIF-1α. This discovery allows for design of new diagnostic tools and novel therapies targeted against angiogenesis-associated diseases, such as cancer. In another aspect, the present invention shows that A_{2A} receptor expression is a marker of the developing lung, and can be used as a marker of lung diseases, such as pulmonary hypertension.

### CROSS REFERENCE TO RELATED APPLICATIONS

This application is a continuation of U.S. application Ser. No. 14/818,557, filed on Aug. 5, 2015, which is a continuation of U.S. application Ser. No. 13/662,898, filed Oct. 29, 2012, now abandoned, which is a continuation of U.S. application Ser. No. 13/087,607, filed on Apr. 15, 2011, now abandoned, which is a continuation of U.S. application Ser. No. 12/271,051, filed on Nov. 14, 2008, now abandoned, which claims benefit to Provisional Application No. 60/987,892, filed on Nov. 14, 2007. The entire disclosures of the prior applications hereinabove are incorporated by reference.

### GOVERNMENT SUPPORT

This invention was supported in part with funding provided by NIH Grant No. P50 HL084923, U01 HL56263 and HL084376 awarded by the National Institutes of Health. The government may have certain rights to this invention.

### REFERENCE TO SEQUENCE LISTING

This application contains a Sequence Listing submitted as an electronic text file named “Sequence Listing.txt”, having a size in bytes of 3 kb, and created on Feb. 3, 2009. The information contained in this electronic file is hereby incorporated by reference in its entirety pursuant to 37 CFR § 1.52(e)(5).

### FIELD OF THE INVENTION

The field of the present invention is angiogenesis, in particular the regulation of angiogenesis by the hypoxia-inducible transcription factor HIF-2α through selective activation of the Adenosine A_{2A} receptor.

### BACKGROUND

Since Folkman's hypothesis that cancers can be treated by targeting angiogenesis (N Engl J Med, 285, 1182 (1971)), there has been a growing interest in targeting the tumor angiogenic pathway. Angiogenesis is a multistep process involving endothelial cell proliferation, migration and invasion resulting in endothelial branching. Growth and progression of solid tumors occurs under conditions of low oxygen (hypoxia) and depends on angiogenesis which provides nutrients to the growing tumor mass and allows for the tumor to metastasize.

One mechanism by which hypoxia promotes tumor growth is via stabilization of hypoxia-inducible transcription factors (HIFs), HIF-1α and HIF-2α. These HIFs recognize the same consensus DNA binding element and regulate common set of genes involved in cell growth, proliferation and angiogenesis, most notable of them being the vascular endothelial growth factor (VEGF).

Although a number of genes are uniquely regulated by HIF-1α in almost all cell types, HIF-2α regulates only a very few unique genes that are limited mainly to specific cell lines. In most cell types, genes regulated by HIF-2α overlap with those of HIF-1α (Semenza et al., Exp Physiol, 91, 803 (2006)). Thus, the role of HIF-2α is not well defined. Prior to the present invention, in a study of non-small cell lung cancer (NSCLC), HIF-2α expression was found to be associated with intense VEGF/KDR-activated vascularization and poor prognosis, whereas HIF-1α expression was marginally associated with poor survival outcome (Giatromanolaki et al., Br J Cancer, 85, 881 (2001)). Although this study underscores the importance of HIF-2α in NSCLC, the mechanisms by which it promotes angiogenesis and tumorigenesis, independent of HIF-1α, remains obscure.

Hypoxia also can cause release of adenosine (Daval et al., Pharmacol Ther 71, 325 (1996); Nees et al., Adv Exp Med Biol 122B, 25 (1979)). Adenosine, a natural ligand for adenosine receptors, has long been known to stimulate angiogenesis through activation of its A₁, A_{2A} A_{2B} or the A₃ receptors. Expression of adenosine receptors is cell and tissue specific. Thus, differential adenosine receptor subtype expression is likely to play an important role in governing cell and tissue specific regulatory pathways in tumor angiogenesis. There is growing evidence that, among the adenosine receptor subtypes, both adenosine A_{2A} and A_{2B} receptors have a more important role in promoting angiogenesis (Feoktisov et al., Hypertension 44, 649 (2002)). The involvement of adenosine A_{2A} receptor in wound healing (Montesinos et al., Am J Pathol 164, 1887 (2004)) also implicates it as an angiogenic regulator. Activation of A_{2A}, but not A_{2B}, receptor promotes angiogenesis in HUVEC (human umbilical vein endothelial cells) and HLMVEC (human lung microvascular endothelial cells) (Desai. et al., Mol Pharmacol 67, 1406 (2005)). Despite these reports, much remains to be understood regarding the precise role of individual adenosine receptors in hypoxia and angiogenesis.

Thus, a thorough understanding of the molecular events involved in HIF-2α mediated angiogenesis and the role of adenosine receptors in this process is needed for the development of better diagnostic tools, as well as for the design of novel anti-angiogenic therapies.

### SUMMARY OF THE INVENTION

In one embodiment, the present invention comprises a method to diagnose a patient with a cancer that is associated with HIF-2α expression, comprising detecting the expression of adenosine A_{2A} receptor (A_{2A}) in a sample of tumor cells from a patient; comparing the level of expression of A_{2A} detected in the patient sample to a level of expression of A_{2A} in a non-tumor cell control sample; and diagnosing the patient as having a cancer that is associated with HIF-2α, if the expression level of A_{2A} in the patient's tumor cells is statistically higher than the expression level of A_{2A} in the non-tumor cell control.

In another embodiment, the present invention comprises a method to identify cancer patients with a poor prognosis for survival comprising: detecting the expression of adenosine A_{2A} receptor (A_{2A}) in a sample of tumor cells from a patient; comparing the level of expression of A_{2A} detected in the patient sample to a level of expression of A_{2A} in a non-tumor cell control sample; and selecting the patient as having a poor prognosis for survival, if the expression level of A_{2A} in the patient's tumor cells is statistically higher than the expression level of A_{2A} in the non-tumor cell control.

In another embodiment, the present invention comprises a method to identify cancer patients with a high level of tumor aggressiveness, comprising: detecting the expression of adenosine A_{2A} receptor (A_{2A}) in a sample of tumor cells from a patient; comparing the level of expression of A_{2A} detected in the patient sample to a level of expression of A_{2A} in a non-tumor cell control sample; and selecting the patient as having a high level of tumor aggressiveness, if the expression level of A_{2A} in the patient's tumor cells is statistically higher than the expression level of A_{2A} in the non-tumor cell control.

In another embodiment, the present invention comprises a method to select a cancer patient who is predicted to benefit from therapeutic administration of a HIF-2α antagonist, an agonist thereof, or a drug having substantially similar biological activity as the HIF-2α antagonist, comprising: detecting the expression of adenosine A_{2A} receptor (A_{2A}) in a sample of tumor cells from a patient; comparing the level of expression of A_{2A} detected in the patient sample to a level of expression of A_{2A} in a non-tumor cell control sample; and selecting the patient as being predicted to benefit from therapeutic administration of the HIF-2α antagonist, if the expression level of A_{2A} in the patient's tumor cells is statistically higher than the expression level of A_{2A} in the non-tumor cell control.

In another embodiment, the present invention comprises a method to select a cancer patient who is predicted to benefit from therapeutic administration of an antagonist of the PI3K/Akt signal transduction pathway, comprising: detecting the expression of adenosine A_{2A} receptor (A_{2A}) in a sample of tumor cells from a patient; comparing the level of expression of A_{2A} detected in the patient sample to a level of expression of A_{2A} in a non-tumor cell control sample; and selecting the patient as being predicted to benefit from therapeutic administration of the HIF-2α antagonist, if the expression level of A_{2A} in the patient's tumor cells is statistically higher than the expression level of A_{2A} in the non-tumor cell control.

In some embodiments, the expression of the A_{2A} is detected by measuring amounts of transcripts of the gene in the tumor cells. In some embodiments the expression of A_{2A} is detected by detecting the A_{2A} protein.

In some embodiments, the non-tumor cell control is a cell of the same type as the tumor cell. In some embodiments, the non-tumor cell control is an autologous, non-cancerous cell from the patient.

In some embodiments, the control expression levels of A_{2A} have been predetermined.

In another embodiment, the present invention comprises a method for in vivo imaging for cancer diagnosis or prognosis, comprising labeling adenosine A_{2A} receptors (A_{2A}) expressed by cells of a patient in vivo, and identifying labeled cells using an imaging method, wherein a high level of labeled cells in the patient, as compared to a normal control, indicates a diagnosis of cancer in the patient, or a poor prognosis for survival in the patient.

In another embodiment, the present invention comprises a method to identify the stage of lung development in a fetus or neonatal infant, comprising detecting adenosine A_{2A} receptor (A_{2A}) expression in the lung cells of the fetus or neonatal infant, wherein detection of a higher level of A_{2A} receptors in the fetus or neonatal infant as compared to a normal control indicates that the lung of the fetus or neonatal infant is undergoing development as compared to the normal control.

In another embodiment, the present invention comprises a method to modulate lung development in a fetus or neonatal infant, comprising modulating the expression or activity of adenosine A_{2A} receptor (A_{2A}) in the lung cells of the fetus or infant. In some embodiments, the infant has respiratory distress syndrome.

In another embodiment, the present invention comprises a method to identify agents that inhibit the development of pulmonary hypertension and related conditions, comprising identifying agents that decrease the expression or activity of adenosine A_{2A} receptor (A_{2A}) in lung cells.

In another embodiment, the present invention comprises a method to inhibit the development of pulmonary hypertension and related conditions, comprising inhibiting the expression or activity of adenosine A_{2A} receptor (A_{2A}) in lung cells of a patient with pulmonary hypertension or a related condition.

In another embodiment, the present invention comprises a method to inhibit angiogenesis in a patient, comprising reducing the activity of the A_{2A} receptor in the patient. In some embodiments the method to inhibit angiogenesis may be used for the treatment of a disease that is associated with an increase in angiogenesis, such as cancer, diabetic blindness, age-related macular degeneration, rheumatoid arthritis, and psoriasis.

In another embodiment, the present invention comprises a method to promote angiogenesis in a patient, by increasing the activity of the A_{2A} receptor in the patient. In some embodiments the method to promote angiogenesis may be used for the treatment of a disease that is associated with insufficient angiogenesis, such as coronary artery disease, stroke, and delayed wound healing.

### BRIEF DESCRIPTION OF THE DRAWINGS

FIGS. 1A, 1B and 1C show that hypoxia and HIF stabilizers regulate the expression of A_{2A} receptor. FIG. 1A is a representative Northern blot that shows that the steady-state mRNA of adenosine A_{2A} receptor, and not the related adenosine A_{2B} receptor, increases when human lung microvascular endothelial cells (HLMVEC) are exposed to hypoxia. FIG. 1B is a representative Western blot that shows that there is an increase in A_{2A} receptor protein, starting at 8 h, when HLMVEC is exposed to hypoxia. FIG. 1C is a representative Northern blot that shows the effect of HIF-stabilizing agents DFO, DMOG and CoCl₂ on A_{2A} receptor expression and demonstrates that HIF stabilization by these agents increases adenosine A_{2A} receptor steady-state mRNA levels in two different donors of ages 14 and 57 years, and in HLMVE cells as well as human coronary artery endothelial cells (HCAEC).

FIGS. 2A and 2B show the effect of adenoviral mutant HIF-1α and mutant HIF-2α on the mRNA levers of A_{2A} receptor. FIG. 2A is a representative Northern blot showing that only HIF-2α, but not HIF-1α, increased adenosine A_{2A} mRNA in HLMVEC in two different donors of ages 11 years and 18 years, as well as in HPAEC. FIG. 2B summarizes the results obtained from a number of experiments and shows that HIF-2α knockdown by using siRNA targeted against HIF-2α, decreased expression of A_{2A} receptor mRNA.

FIGS. 3A, 3B and 3C illustrate the transcriptional regulation of A_{2A} receptor by HIF-2α. FIG. 3A shows that in 293 cells and HLMVE cells pSSG-luciferase reporter vectors carrying the putative promoter R5 from the promoter region of A_{2A} receptor, showed an increase in luciferase activity when co-transfected with a mutated, constitutively active HIF-2α construct. FIG. 3B (SEQ ID NO:1) shows the sequence of the R5 promoter; hypoxia response elements in the R5 promoter are shown in bold and primers used in amplifying the hypoxia response element are underlined. FIG. 3C shows that there is an in vivo association of the endogenously active HIF-2α with hypoxia-responsive element within the A_{2A} receptor promoter, by presenting the results of the chromatin immunoprecipitation (ChIP) assays. Immunoprecipitation of the chromatin complexes formed when HLMVEC were exposed to hypoxia showed significant enrichment of the A_{2A} promoter fragment with the specific HIF-2α antibody when compared to the normoxic control or the mock antibody control. Similar enrichment of PGK-1 was also observed in HLMVEC under identical conditions and was used as a positive control.

FIGS. 4A, 4B, 4C and 4D show that activation or expression of A_{2A} receptor promotes cellular proliferation, migration and branching. FIG. 4A shows that activation of adenosine A_{2A} receptor by exposure to the A_{2A} receptor agonist CGS-21680 significantly increased cellular proliferation as assessed by ³[H]thymidine incorporation in a dose-dependent manner. FIG. 4B shows that expression of A_{2A} receptor using an adenoviral vector significantly increased cellular proliferation as assessed by ³[H]thymidine incorporation when compared to control non-transduced cells or the Ad.LacZ-transduced cells. FIG. 4C shows that expression of A_{2A} receptor promotes endothelial cell migration, by showing the increase in migration of HLMVEC across a fibronectin-coated membrane in response to increased A_{2A} receptor expression; there was increased migration of cells transduced with Ad.A_{2A} compared to both the Ad.LacZ control and the non-transduced control. FIG. 4D shows that activation of adenosine A_{2A} receptor by exposure to the agonist CGS-21680 promotes endothelial sprouting or branching in HLMVEC relative to control cells.

FIG. 5 shows the effect of HIF-1α and HIF-2α on the expression of hexokinase-II (HK2) and VEGFA. Both HIF-1α and HIF-2α transcriptionally upregulated VEGFA, but only HIF-1α upregulated HK2.

FIG. 6 shows that both HIF-1α and HIF-2α knockdowns decreased VEGF mRNA levels in HLMVEC.

FIG. 7 shows that in contrast to human derived endothelial cells, in mouse derived endothelial cells SVEC and MB114, neither hypoxia nor HIF stabilization by DMOG altered the expression of A_{2A} receptor mRNA levels in HLMVEC.

FIG. 8 is a representative Northern blot that shows the effect of HIF-2α on the mRNA levels of A_{2A} receptor. It shows that only HIF-2α regulates A_{2A} receptor expression, while both HIF-1α and HIF-2α regulate VEGF and only HIF-1α regulates hexokinase-II (HKII).

FIG. 9 shows that Adenosine A_{2A} receptor activation by exposure to the agonist CGS-21680 promotes tube formation in a dose dependent manner.

FIG. 10 shows the expression of Adenosine A_{2A} receptor in different tumor stages of the cancer.

FIGS. 11A and 11B show that siRNA targeted against A_{2A} is able to knock down the expression of A_{2A}. FIG. 11A represents a Northern blot showing A_{2A} receptor expression in HLMVEC where the cell is transduced with an adenoviral vector carrying the A_{2A} receptor gene.

FIG. 11B represents a Northern blot showing that co-expression of siRNA targeted against A_{2A} in a transient transfection assay knocks down the expression of the A_{2A}.

FIG. 12 shows that activation of the A_{2A} receptor by exposure to agonist increases PI 3-kinase activity in HLMVECs. The left panel is a representative autoradiogram demonstrating that activation of the A_{2A} receptor by exposure to agonist increases PI 3-kinase-mediated phosphorylation of phosphoinositides, PIP3. The right panel is a representative Western blot demonstrating that that activation of the A_{2A} receptor by exposure to agonist increases expression of phosphorylated Akt (a downstream target of PI 3-Kinase).

FIG. 13 shows the pattern of A_{2A} and A_{2B} receptor expression in maturing baboon lung. The upper panel contains representative Northern blots showing RNA from gestational control (GC), Gestational control born prematurely and provided oxygen as needed (PRN) and Term baboons hybridized with probes for A_{2A} and A_{2B} and autoradiographed and shows that A_{2A} receptor expression is higher in the lung undergoing development and decreases as the lung nears full development. The lower panel, left graph shows the quantification of the A_{2A} and A_{2B} receptor RNA bands and plots the relative intensity of the bands using 28S RNA as control. The lower panel, right graph shows the PI3-Kinase activity corresponding to the 125, 140 and 160 d.g.c.

FIG. 14 is a schematic representation of a proposed model for regulation of A_{2A} receptor and its function.

### DETAILED DESCRIPTION

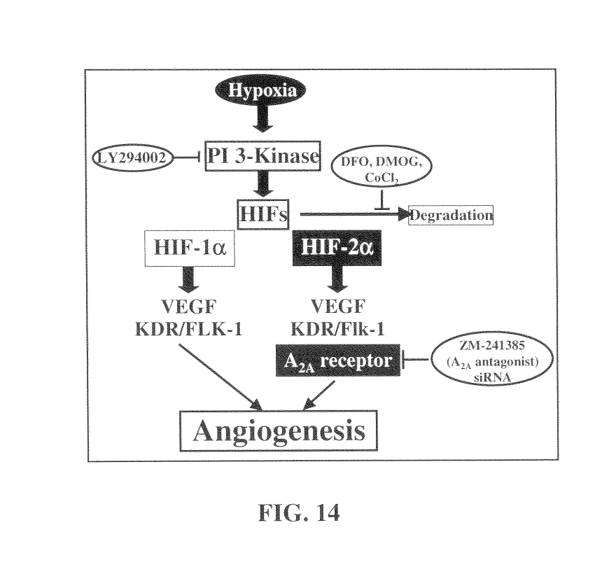

This invention generally relates to the discovery by the inventors that HIF-2α, but not HIF-1α, selectively regulates adenosine A_{2A} receptor (also referred herein as A_{2A} receptor or ADORA2A) in endothelial cells, thereby revealing a unique pathway by which HIF-2α can regulate angiogenesis independent of HIF-1α. (FIG. 14 shows a schematic representation of the proposed model.)

The inventors show herein that overexpression of A_{2A} or its activation increases endothelial cell proliferation and angiogenesis. Therefore, A_{2A} is an angiogenic marker of HIF-2α activation in the microvasculature of the human lung that promotes tumor growth and neovascularization, and is a potential new target for anti-angiogenic therapy in lung cancer. The invention also sets forth A_{2A} as a powerful marker for diagnosing cancer patients, and perhaps more significantly, for identifying patients with aggressive tumors and/or a poor prognosis for survival. Such a prognosis thereby reveals those patients for whom personalized therapy via specific targeting of pathways associated with HIF-2α and A_{2A} may be especially useful. The inventors provide evidence herein that an A_{2A} agonist increases PI 3-kinase activity in human lung microvascular endothelial cells (HLMVECs), indicating that patients with tumors expressing higher than normal levels of A_{2A} are candidates for cancer therapy that target this signal transduction pathway (i.e., via PI 3-kinase, PIP3, Akt, etc.).

Using adenoviral mutHIF-1α and adenoviral mutHIF-2α constructs, where HIFs are transcriptionally active under normoxic conditions, it is shown here that VEGF and its receptor Flt1, are regulated by both HIFs in primary lung endothelial cells including those from the microvasculature. However, only HIF-2α regulates adenosine A_{2A} receptor (A_{2A}) in these endothelial cells. Previous studies have shown that A_{2A} can be angiogenic. Angiogenesis is a multistep process involving endothelial cell proliferation, migration and invasion resulting in endothelial branching. In the present study, activation of A_{2A} by specific agonist, CGS21680, increased cellular proliferation in a dose-dependent manner, as assessed by ³[H]-thymidine incorporation. Cellular proliferation also increased by 2.5-fold when A_{2A} was overexpressed using an adenoviral-mediated system. Similarly, A_{2A} overexpressing cells exhibited a 3-fold increase in cell migration when compared to the non-transduced or the Ad.LacZ transduced controls. Further, endothelial branching using a Matrigel-matrix based assay was assessed. In presence of the A_{2A} agonist, CGS21680, there was a 37% increase in branching when compared to the diluent control. These data demonstrate a unique pathway by which HIF-2α can regulate angiogenesis independent of HIF-1α.

Accordingly, the present invention relates to methods to diagnose a patient with a cancer that is associated with HIF-2α expression, to identify cancer patients with a poor prognosis for survival, to identify cancer patients with a high level of tumor aggressiveness, to select a cancer patient who is predicted to benefit from therapeutic administration of a HIF-2α antagonist, and to select a cancer patient who is predicted to benefit from therapeutic administration of an antagonist of the PI3K/Akt signal transduction pathway.

These methods generally include detecting a level of expression of adenosine A_{2A} receptor (A_{2A}) in a sample of tumor cells from a patient and comparing this level of expression to a control level of expression (e.g., in a non-tumor cell control sample). Positive controls may also be used for comparison. Patients are then selected on the basis of whether the expression of A_{2A} in their tumors is higher than in a non-cancerous cell, or alternatively similar to a tumor with a known positive correlation with HIF-2α. Patients with higher levels of A_{2A} expression are identified as having tumors associated with HIF-2α, which not only improves the specificity of the diagnosis of cancer, but also indicates a poor survival prediction and a high tumor aggressiveness for the patient. Such patients may then be candidates for a more “personalized” therapeutic approach, since drugs and therapies that are not predicted to be useful for such cancers can be eliminated from consideration, and more importantly for the patient, drugs and therapies that specifically target the HIF-2α and/or A_{2A} pathways may be selected as particularly useful for such patients. Accordingly, the discovery by the inventors represents a new marker for diagnosis and design of a personalized medical therapy for certain cancer patients.

In one embodiment of the invention, A_{2A} is used as an in vivo imaging marker for cancer prognosis, tumor aggressiveness and/or therapeutic approach selection. In this aspect of the invention, a tagged (i.e., fluorescent or radiolabeled or other imaging tag) protein or probe is used to bind to cells with accessible A_{2A} in vivo. Tagged cells can then be followed by identifying such cells on histological sections, positron emission tomography (PET) imaging, ultrasound, or other known techniques.

In another embodiment, the present invention includes a method to inhibit angiogenesis, by reducing the activity of the A_{2A} receptor in the cells. The term “reducing activity” as used herein includes reducing the activity by at least about 5%, and more preferably at least about 10%, and more preferably at least 20%, and more preferably at least 25%, and more preferably at least 30%, and more preferably at least 35%, and more preferably at least 40%, and more preferably at least 45%, and more preferably at least 50%, and preferably at least 55%, and more preferably at least 60%, and more preferably at least 65%, and more preferably at least 70%, and more preferably at least 75%, and more preferably at least 80%, and more preferably at least 85%, and more preferably at least 90%, and more preferably at least 95%, and more preferably of 100%, of the level of activity of A_{2A} in the cell.

The activity may be reduced by using molecules that specifically target the A_{2A} receptor protein and inhibit its activity. Such molecules may include, without limitation, drugs, chemicals, ligands, inhibitors, antagonists, competitors, peptides or proteins that bind to the A_{2A} receptor. The activity may be reduced by reducing the expression of the A_{2A} receptor protein. Techniques for reducing expression of the protein may include, without limitation, antisense RNA, use of transcriptional or translational inhibitors, and gene knock-out technology.

The method to inhibit angiogenesis may be used to treat any angiogenesis-associated or angiogenesis-dependent disease, which shows an increase in angiogenesis. Such diseases may include, without limitation, cancer, diabetic blindness, age-related macular degeneration, rheumatoid arthritis, or psoriasis.

Conversely, in another embodiment the present invention may include a method to promote angiogenesis, by increasing the activity of the A_{2A} receptor in the cells. The activity may be increased by using molecules that specifically target the A_{2A} receptor protein to activate it such as, without limitation, drugs, chemicals, ligands, or agonists. The activity may be increased by increasing the expression of the A_{2A} receptor protein by using expression vectors carrying the gene for the receptor protein or by the activation of the HIF-2α pathway. Such method may be used to treat diseases that are associated with insufficient angiogenesis, such as coronary artery disease, stroke, and delayed wound healing.

In another aspect of the invention, the inventors have discovered that A_{2A} expression is a marker of the developing lung, and can also be used as a marker of lung diseases, such as pulmonary hypertension. Referring to data provided herein, the inventors demonstrate that A_{2A} expression is higher in the lung undergoing development and decreases as the lung nears full development. Accordingly, one embodiment of the invention relates to the targeting of A_{2A} (e.g., by modulating the expression or activity of A_{2A} or a downstream molecule in the pathway) to modulate lung development, for example, in preterm infants, or in infants with respiratory distress syndrome (RDS). Another embodiment relates to the use of A_{2A} as a marker for identification of fetal or neonatal lung development, in that fetuses and neonates with higher levels of A_{2A} expression may still be undergoing lung development than counterparts with lower levels of A_{2A} expression. On the other hand, loss of HIF-2α has been associated with RDS, and so excessively low expression of A_{2A} may also serve to identify such patients.

In another embodiment, A_{2A} can be used as a therapeutic target for the treatment of pulmonary hypertension. Chronic hypoxic conditions are known to induce pulmonary vascular remodeling and subsequent pulmonary hypertension and right ventricular hypertrophy, thereby constituting a major cause of morbidity and mortality in patients with chronic obstructive pulmonary disease (COPD). HIF-2α was previously proposed to be a marker for screening molecules that are able to inhibit the development of pulmonary hypertension, and HIF-2α inhibitors have been proposed for the treatment of pulmonary hypertension. However, given the discovery of the present invention, the more accessible and easily targeted A_{2A} is set forth to be a marker for screening molecules that are able to inhibit the development of pulmonary hypertension, and A_{2A} inhibitors are now proposed for the treatment of pulmonary hypertension.

Various definitions and aspects of the invention will be described below, but the invention is not limited to any specific embodiments that may be used for illustrative or exemplary purposes.

Tumor aggressiveness is defined herein as an ability of or propensity of a tumor to metastasize, as well as the ability to grow beyond a critical size (e.g., about 2-3 mm). Tumors larger than this approximate size typically require vascularization.

As used herein, the term “expression”, when used in connection with detecting the expression of A_{2A}, can refer to detecting transcription of the gene (i.e., detecting mRNA levels) and/or to detecting translation of the gene (detecting the protein produced). To detect expression of a gene refers to the act of actively determining whether a gene is expressed or not. This can include determining whether the gene expression is upregulated as compared to a control, downregulated as compared to a control, or unchanged as compared to a control. Therefore, the step of detecting expression does not require that expression of the gene actually is upregulated or downregulated, but rather, can also include detecting that the expression of the gene has not changed (i.e., detecting no expression of the gene or no change in expression of the gene).

Expression of transcripts and/or proteins is measured by any of a variety of known methods in the art. For RNA expression, methods include but are not limited to: extraction of cellular mRNA and Northern blotting using labeled probes that hybridize to transcripts encoding all or part of A_{2A}; amplification of mRNA using A_{2A}-specific primers, polymerase chain reaction (PCR), and reverse transcriptase-polymerase chain reaction (RT-PCR), followed by quantitative detection of the product by any of a variety of means; extraction of total RNA from the cells, which is then labeled and used to probe cDNAs or oligonucleotides encoding A_{2A} on any of a variety of surfaces; in situ hybridization; and detection of a reporter gene.

Methods to measure protein expression levels generally include, but are not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry, as well as assays based on a property of the protein including but not limited to enzymatic activity or interaction with other protein partners. Binding assays are also well known in the art. For example, a BIAcore machine can be used to determine the binding constant of a complex between two proteins. The dissociation constant for the complex can be determined by monitoring changes in the refractive index with respect to time as buffer is passed over the chip (O'Shannessy et al. Anal. Biochem. 212:457 (1993); Schuster et al., Nature 365:343 (1993)). Other suitable assays for measuring the binding of one protein to another include, for example, immunoassays such as enzyme linked immunoabsorbent assays (ELISA) and radioimmunoassays (RIA); or determination of binding by monitoring the change in the spectroscopic or optical properties of the proteins through fluorescence, UV absorption, circular dichroism, or nuclear magnetic resonance (NMR). A preferred method is an immunoassay, wherein an A_{2A}-specific antibody (an antibody that selectively binds to A_{2A}) is used to detect the expression on tumor cells.

A patient sample can include any bodily fluid or tissue from a patient that may contain tumor cells or proteins of tumor cells. More specifically, according to the present invention, the term “test sample” or “patient sample” can be used generally to refer to a sample of any type which contains cells or products that have been secreted from cells to be evaluated by the present method, including but not limited to, a sample of isolated cells, a tissue sample and/or a bodily fluid sample. According to the present invention, a sample of isolated cells is a specimen of cells, typically in suspension or separated from connective tissue which may have connected the cells within a tissue in vivo, which have been collected from an organ, tissue or fluid by any suitable method which results in the collection of a suitable number of cells for evaluation by the method of the present invention. The cells in the cell sample are not necessarily of the same type, although purification methods can be used to enrich for the type of cells that are preferably evaluated. Cells can be obtained, for example, by scraping of a tissue, processing of a tissue sample to release individual cells, or isolation from a bodily fluid.

Preferably, a level of expression of A_{2A} identified as being upregulated (overexpressed, expressed at a higher level than in a normal cell) in a tumor cell according to the invention is upregulated at least about 5%, and more preferably at least about 10%, and more preferably at least 20%, and more preferably at least 25%, and more preferably at least 30%, and more preferably at least 35%, and more preferably at least 40%, and more preferably at least 45%, and more preferably at least 50%, and preferably at least 55%, and more preferably at least 60%, and more preferably at least 65%, and more preferably at least 70%, and more preferably at least 75%, and more preferably at least 80%, and more preferably at least 85%, and more preferably at least 90%, and more preferably at least 95%, and more preferably of 100%, or any percentage change between 5% and higher in 1% increments (i.e., 5%, 6%, 7%, 8% . . . ), of the level of expression of A_{2A} that is seen in normal, non-cancerous cells, or even in tumor cells not associated with HIF-2α. The values obtained from the test (tumor) and/or control samples are statistically processed using any suitable method of statistical analysis to establish a suitable baseline level using methods standard in the art for establishing such values. Statistical significance according to the present invention should be at least p<0.05.

The presence and quantity of A_{2A} can be measured in primary tumors, metastatic tumors, locally recurring tumors, ductal carcinomas in situ, or other tumors. The markers can be measured in solid tumors that are fresh, frozen, fixed or otherwise preserved.

The level of expression of A_{2A} detected in the patient sample is compared to a baseline or control level of expression of A_{2A}. More specifically, according to the present invention, a “baseline level” is a control level of A_{2A} expression against which a test level of A_{2A} expression (i.e., in the test sample) can be compared. In the present invention, the control level of A_{2A} expression can be the expression level of A_{2A} in a control cell that is normal (non-tumor) and/or the expression level of A_{2A} in a control cell that is positive for HIF-2α association. Other controls may also be included in the assay. In one embodiment, the control is established in an autologous control sample obtained from the patient. The autologous control sample can be a sample of isolated cells, a tissue sample or a bodily fluid sample, and is preferably a cell sample or tissue sample. According to the present invention, and as used in the art, the term “autologous” means that the sample is obtained from the same patient from which the sample to be evaluated is obtained. The control sample should be of or from the same cell type and preferably, the control sample is obtained from the same organ, tissue or bodily fluid as the sample to be evaluated, such that the control sample serves as the best possible baseline for the sample to be evaluated. In one embodiment, control expression levels of A_{2A} has been predetermined. Such a form of stored information can include, for example, but is not limited to, a reference chart, listing or electronic file of A_{2A} expression levels. Therefore, it can be determined, based on the control or baseline level of A_{2A} expression or biological activity, whether the expression level of A_{2A} in a patient sample is more statistically significantly similar to the baseline for HIF-2α association or to a normal, non-tumor cell (or a tumor cell that is not associated with HIF-2α expression).

Isolated antibodies useful in the present invention can include serum containing such antibodies, or antibodies that have been purified to varying degrees. Whole antibodies can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), may also be employed in the invention.

Limited digestion of an immunoglobulin with a protease may produce two fragments. An antigen binding fragment is referred to as an Fab, an Fab′, or an F(ab′)₂ fragment. A fragment lacking the ability to bind to antigen is referred to as an Fc fragment. An Fab fragment comprises one arm of an immunoglobulin molecule containing a L chain (V_{L}+C_{L} domains) paired with the V_{H} region and a portion of the C_{H} region (CH1 domain). An Fab′ fragment corresponds to an Fab fragment with part of the hinge region attached to the CH1 domain. An F(ab′)2 fragment corresponds to two Fab′ fragments that are normally covalently linked to each other through a di-sulfide bond, typically in the hinge regions.

According to the present invention, the phrase “selectively binds to” refers to the ability of an antibody, antigen binding fragment or binding partner (antigen binding peptide) to preferentially bind to specified proteins. More specifically, the phrase “selectively binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.).

Agonists and antagonists that are products of drug design can be produced using various methods known in the art. Various methods of drug design, useful to design mimetics or other compounds useful in the present invention are disclosed in Maulik et al., 1997, Molecular Biotechnology: Therapeutic Applications and Strategies, Wiley-Liss, Inc., which is incorporated herein by reference in its entirety. An agonist or antagonist can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the similar building blocks) or by rational, directed or random drug design. See for example, Maulik et al., supra.

### EXAMPLES

### Example 1: This Example Illustrates that Hypoxia and HIF Stabilizers Regulate the Expression of Adenosine Receptor A_{2A}

Primary human lung microvascular endothelial cells (HLMVEC), primary human coronary artery endothelial cells (HCAEC), primary pulmonary artery endothelial cells (HPAEC) and endothelial cell growth medium were obtained from Cambrex (Walkersville, Md.). Dimethyloxlylglycine (DMOG) was obtained from Frontier Scientific, Inc (Logan, Utah). HLMVEC and HCAEC were cultured in endothelial cell basal medium (EBM-2) supplemented with VEGF, human FGF, human EGF, hydrocortisone, ascorbic acid, insulin-like growth factor-1, GA-1000 (gentamycin/amphotericin-B), 5% fetal bovine serum as per the supplier's protocol. Murine brain microvascular endothelial cells (MB114) and SV-40 transformed mouse endothelial cells (SVEC) were cultured in DMEM supplemented with 10% FBS, penicillin and streptomycin. The same culture conditions were used in subsequent examples, unless specifically noted otherwise.

To assess the role of adenosine receptors in hypoxia, primary HLMVEC were exposed to air or hypoxia. For detecting mRNA, twenty-four hours post-transduction or treatment, cells were washed twice with Hank's balanced salt solution (HBSS) and harvested in GITC. RNA was purified using the CsCl method as described earlier (Riddle et al., Am J Physiol Lung Cell Mol Physiol 278, L407 (2000). 15 μg total RNA were resolved on 1% formaldehyde-agarose gels and transferred to nylon membranes. Probes used for northern blot were derived from human A_{2A} cDNA and human A_{2B} cDNA kindly provided by Dr. Marlene Jacobson, Merck Research Labs, West Point, Pa. The VEGF cDNA was obtained from the Harvard Proteomic Institute. The cDNA probes were labeled with [³²P]α-dCTP (ICN, Irvine, Calif.) by random priming and hybridized with the membrane for 18 hrs at 42° C. Membranes were then washed and autoradiographed. For loading controls, membranes were stripped of radioactive probe in a 2% glyceraldehyde solution at 80° C. and rehybridized with an end-labeled 28S rRNA oligonucleotide (Ambion, Austin, Tex.). The intensity of the radiolabeled bands was measured using a PhosphorImager running ImageQuant software (Molecular Dynamics, Sunnyvale, Calif.). The same Northern Blot procedure was used in subsequent examples, unless specifically noted otherwise. Protein expression was detected using standard Western Blot methods described in Ahmad, Free Radic Biol Med. 40(7):1108(2006).

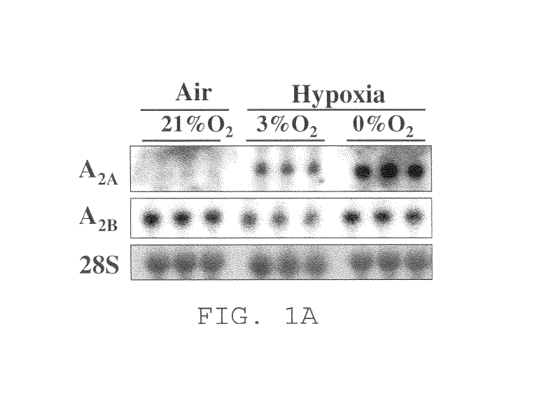

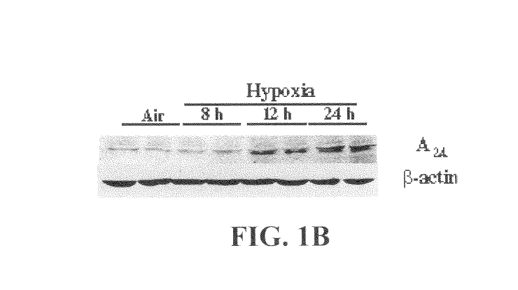

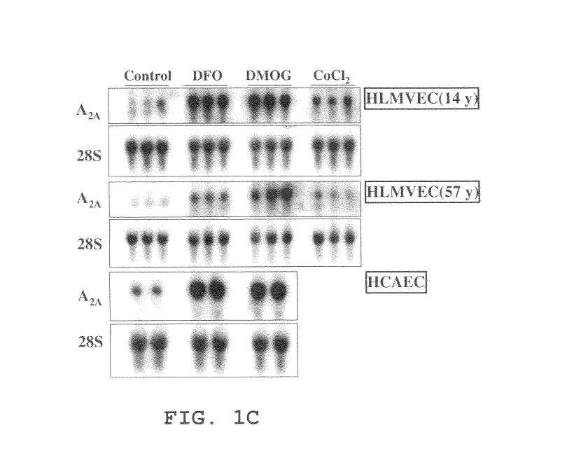

As shown in FIG. 1A, steady-state mRNA of adenosine A_{2A} receptor, and not the related adenosine A_{2B} receptor, increased when HLMVEC were exposed to hypoxia. Correspondingly, as shown in FIG. 1B, there was also an increase in A_{2A} receptor protein, starting at 8 h, when HLMVEC was exposed to hypoxia. Since hypoxic regulation of a large number of genes is mediated by HIF-1α and HIF-2α, the effect of HIF-stabilizing agents DFO, DMOG and CoCl₂ was studied at concentrations that have been previously demonstrated to stabilize both HIF-1α and HIF-2α (Asikainen et al., Free Radic Biol Med 38, 1002 (2005). FIG. 1C demonstrates that HIF stabilization increased adenosine A_{2A} receptor steady-state mRNA levels. This HIF mediated regulation of A_{2A} mRNA level was not restricted to one donor or one endothelial cell type like the HLMVEC but was consistently present in a number of donors and endothelial cells from other sources like the coronary artery as well.

### Example 2: This Example Illustrates that HIF-2α, not HIF-1α, Regulates the Expression of the A_{2A} Receptor

To dissect the role of individual HIFs in regulating adenosine A_{2A} receptor in primary human endothelial cells, adenoviral vectors encoding mutant-HIF-1α or mutant-HIF-2α were constructed. The HIF-1α construct containing mutations at P564A and N803A that allow the protein to be stable and constitutively active under normoxic conditions was obtained from Dr. Murray Whitelaw, Univ. of Adelaide, Australia. An additional mutation was generated at P40_{2A} to prevent any ubiquitylation and subsequent degradation of the HIF-1α protein (Masson et al., Embo J 20, 5197, (2001). The construct was then subcloned into an adenoviral shuttle vector (pShuttle-CMV) using the restriction sites KpnI/XbaI. An adenovirus vector encoding the mutant HIF-1α (Ad. mutHIF1α) was generated using standard procedures. Briefly, the plasmid was linearized using PmeI and used to transform E. coli strain BJ5183 carrying the plasmid AdEasy-1 (He et al., Proc Natl Acad Sci USA 95, 2509 (1998)) to generate the recombinant plasmids containing the entire vector chromosome Recombinant vector DNA encoding the mutant HIF-1α was released from the plasmid by digestion with PacI and used to transfect 293 cells to generate Ad.mutHIF-1α. The vector was plaque purified, grown in large scale, and purified using CsCl step- and isopycnic gradient centrifugation. Ad.mutHIF-2α, encoding the mutant human HIF-2α construct (also from Dr. Whitelaw) containing mutations at P531A and N847A was similarly generated. For generation of an adenovirus vector encoding A_{2A} receptor (Ad.A_{2A}) human adenosine A_{2A} receptor (a kind gift from Dr. Marlene A Jacobson, Merck Research Laboratories, PA) cDNA was excised from pSVL plasmid using XhoI and BamH1 (blunted) and subcloned into the adenoviral shuttle vector pShuttle-CMV using the restriction sites XhoI and EcoRV. Ad.A_{2A} was generated following the protocols outlined above. Adenoviral transductions of HLMVEC were carried out at a multiplicity of infection of 10 plaque forming units per cell as described earlier (Ahmad et al., 2006). For transduced controls Ad.LacZ was used (Schaack et al., J Virol 69, 3920, (1995).

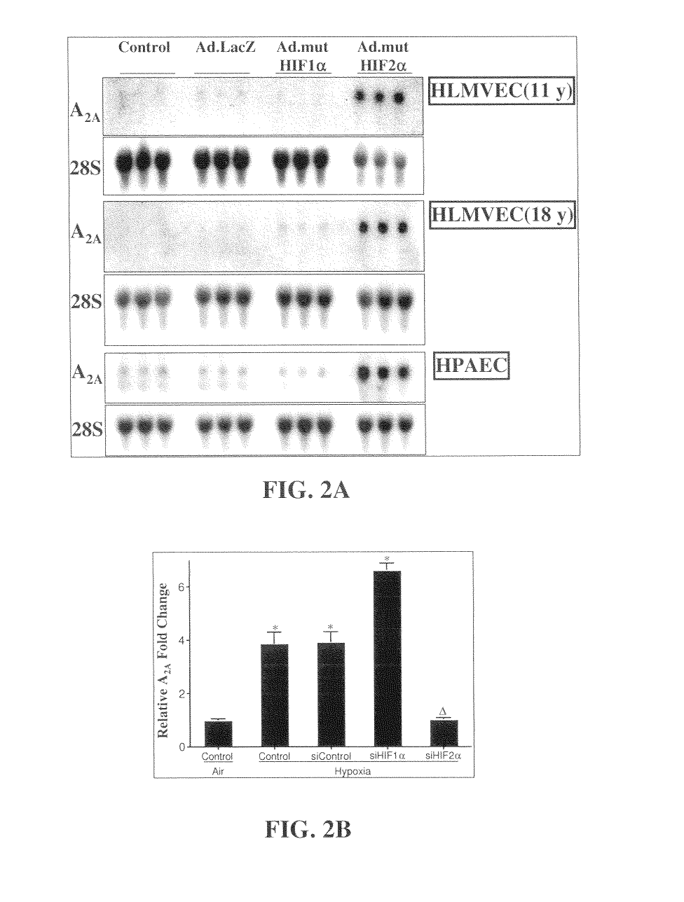

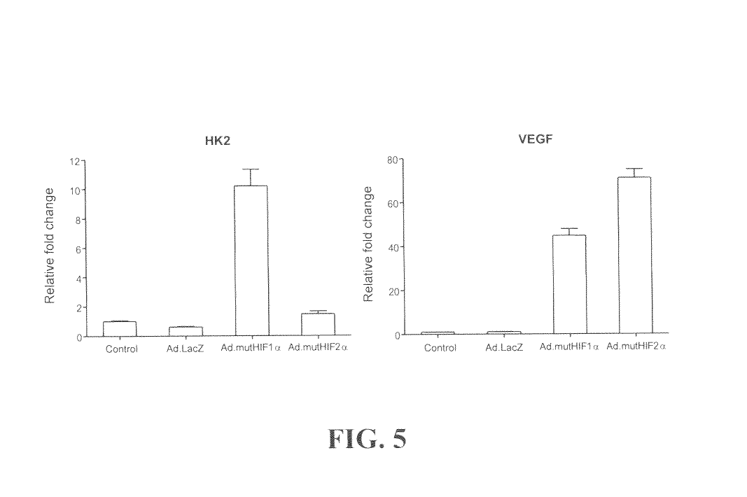

These mutant HIFs were both stable and transcriptionally active under normoxic conditions. As shown in FIG. 5, both HIF-1α and HIF-2α transcriptionally upregulated VEGF, but only HIF-1α upregulated hexokinase-II. Interestingly, only HIF-2α increased adenosine A_{2A} mRNA in primary endothelial cells derived from lung (HLMVEC and HPAEC; FIG. 2A). This HIF-2α specific regulation of A_{2A} receptor was reproducible in at least three different donors of HLMVEC, of which two are shown in FIG. 2. In addition to microvascular endothelial cells, endothelial cells from the macrovessel (HPAEC) also showed similar regulation. The contribution of HIF-1α in upregulating adenosine A_{2A} receptor mRNA was negligible (FIG. 2A).

To further elucidate the physiological role of HIF-2α in regulating A_{2A} receptor, knock downs of HIF-1α and HIF-2α was effected. The knockdowns of HIF-1α and HIF-2α in HLMVEC were carried out using predesigned SmartPool siRNA purchased from Dharmacon. HLMVEC cells were transfected with siRNA against HIF-1α, HIF-2α or the non-targeting control siRNA and exposed to hypoxia. Initially transfection efficiencies were optimized using siGLO Green as an indicator. Transfections were carried out in 6-well plates using 25 nM siRNA complexed to 3 μl of DharmaFect1 transfection reagent in a total volume of 2.0 ml, as per the manufacturers protocol. Twenty four hours post transfection, cells were exposed to hypoxia (0% 02, 5% CO₂, balance N₂) for an additional 24 h, following which RNA was isolated and Real-Time RT-PCR performed using Taqman primers and probes for adenosine A_{2A} receptor, HK2 and VEGFA.

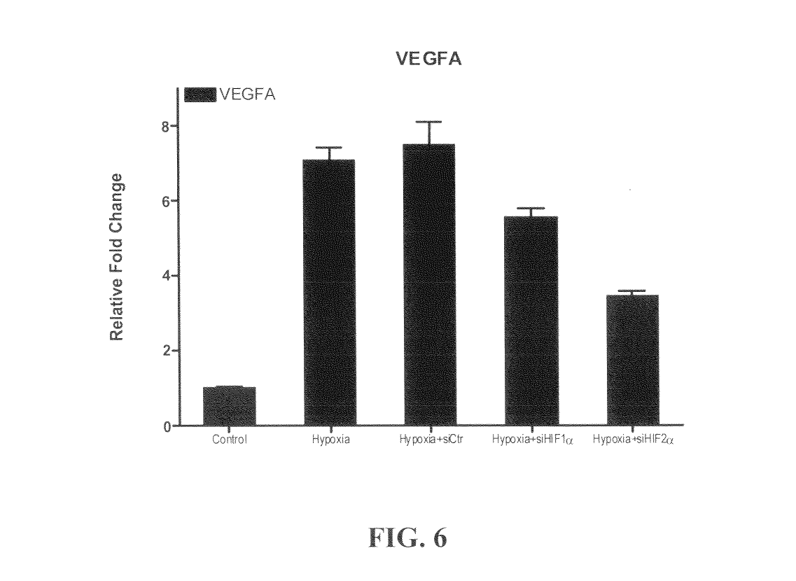

As shown in FIG. 2B, hypoxia increased A_{2A} receptor expression and HIF-2α knockdown reversed this change. As expected, the non-targeting controls did not change expression of the receptor under hypoxic conditions. Also, as shown in FIG. 6, both HIF-1α and HIF-2α knockdowns decreased VEGF expression. Interestingly, HIF-1α knockdown increased A_{2A} receptor (FIG. 2b).

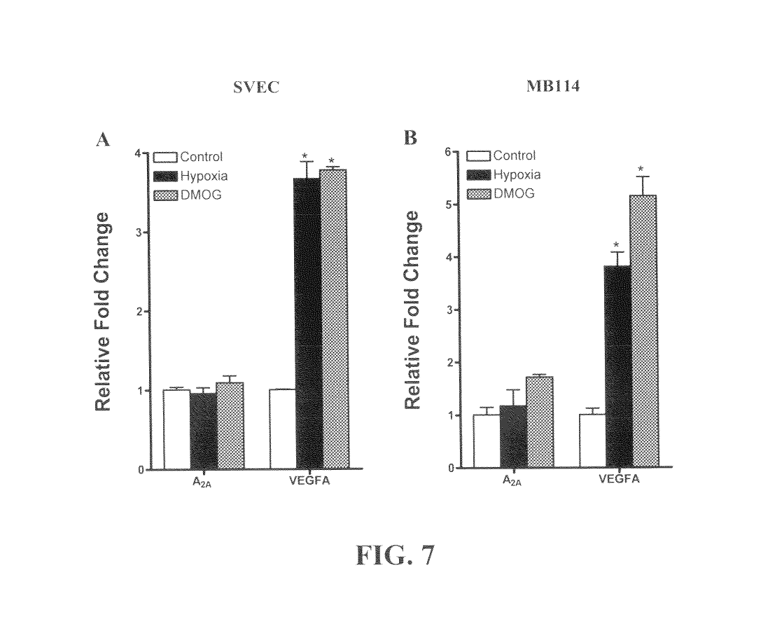

In all cases siRNA transfection efficiencies reached 95% as assessed using siGLO (Dharmacon) as an indicator. These findings in primary human derived endothelial cells were in contrast to those using mouse derived endothelial cells, SVEC and MB114, where hypoxia or HIF stabilization did not alter the expression of A_{2A} receptor (FIG. 7).

All statistical analyses in this Example as well as the subsequent examples were performed with the JMP software (SAS Institute, Cary, N.C., USA). Data are represented as mean±SEM of n>_3 and were compared by ANOVA followed by Tukey-Kramer test for multiple comparisons. A p value of <0.05 was considered significant.

### Example 3: This Example Illustrates the Mechanism of Transcriptional Regulation of the A_{2A} Receptor by HIF-2α

To further evaluate the transcriptional regulation of A_{2A} receptor by HIF-2α, the promoter region of this receptor was analyzed. Earlier bioinformatics analysis of the human A_{2A} receptor gene suggested presence of multiple promoters (Yu et al., Brain Res 1000, 156 (2004)). Putative promoters upstream of the A_{2A} receptor gene were determined as described (Trinklein et al., Genome Research 13, 308 (2003). These promoter constructs, cloned in luciferase reporter constructs were obtained from SwitchGear Genomics (Menlo Park, Calif.). Five of these putative promoters, R1, R2, R3, R5 and R6, were cloned in pSSG-luciferase reporter vectors. Promoter activity of the A_{2A} receptor gene was assessed using a luciferase reporter construct, R5. HLMVECs were transfected with the A_{2A} reporter vectors or the empty control (pGL4.11) together with mutHIF-2α construct using the DharmaFect Duo transfection reagent. In all cases a CMV-β-gal plasmid co-transfection was used to control for transfection efficiency. Forty hours post transfection, cells were harvested and lysed using the reporter lysis buffer (Promega). β-galactosidase assays were performed with a commercially available kit (Stratagene, La Jolla, Calif.). Luciferase activities were determined with a commercially available luciferase assay system (PharMingen, San Diego, Calif.) and a Monolight 3010 luminometer (Analytical Luminescence Laboratory, Cockeyville, Md.). The relative luciferase units were normalized to the internal β-galactosidase control values and plotted. All promoter-luciferase experiments were done in triplicate.

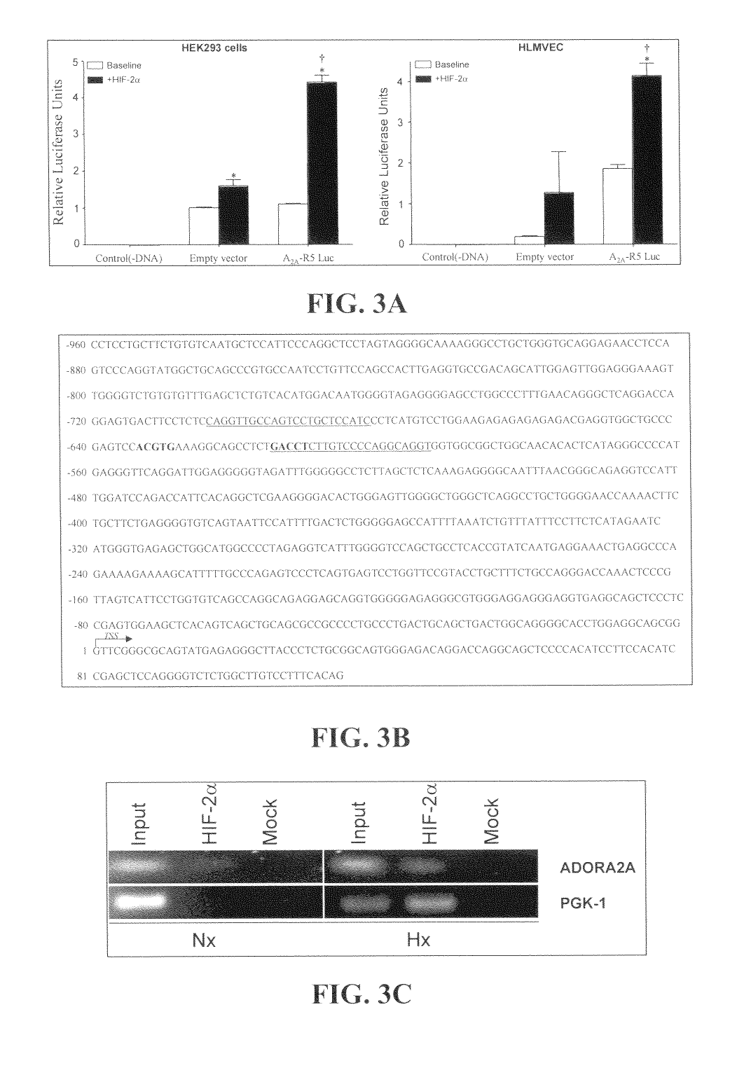

Out of the five putative promoters, R5 showed consistent induction when co-transfected with the HIF-2α constructs. As shown in FIG. 3A, both 293 cells and HLMVEC showed a similar increase in luciferase activity when co-transfected with a mutated, constitutively active HIF-2α construct.

FIG. 3B shows the sequence of the R5 promoter. The primers used in amplifying the hypoxia response element in the R5 promoter are underlined, whereas the hypoxia response elements are shown in bold (FIG. 3B).

To further examine in vivo association of the endogenously active HIF-2α with hypoxia-responsive element within the A_{2A} receptor promoter, chromatin immunoprecipitation (ChIP) assays were performed on HLMVECs using standard protocol. About 45 million cells in 100 mm plates were exposed to air (21% O₂) or hypoxia (1% O₂) for 6 h. Following hypoxic exposure, cells were washed with PBS and crosslinked in a solution of 10% formaldehyde with gentle shaking for 20 min. The crosslinking was stopped by the addition of glycine to a final concentration of 0.125M. Cells were then washed with cold PBS, scraped and pelleted. The pellet was resuspended in lysis buffer (50 mM Tris-HCl pH 8.1 containing 1% SDS, 5 mM EDTA and Calbiochem protease inhibitor cocktail) for 10 min after which the samples were sonicated for 15 sec a total of five times, using a Branson Sonicator. After clearing the lysate, a part of the soluble chromatin was diluted 5-fold in PBS and reverse cross-linked at 65° C. overnight for use as an input control. The remaining soluble chromatin was diluted 10 fold with the dilution buffer (20 mM Tris, pH 8.1, 2 mM EDTA, 1% Triton-X100) and precleared with Protein G beads. The samples were incubated at 4° C. overnight with either a control antibody or rabbit polyclonal antibody against HIF-2α (Novus Biologicals). The chromatin immunoprecipitated DNA was PCR amplified using specific primers for A_{2A} receptor (Forward: 5′-CAGGTTGCCAGTCCTGCTCCATC (SEQ ID NO:2) and Reverse: ACCTGCCTGGGGACAAGAGGTC-3′ (SEQ ID NO:3)) and PGK-1 (Forward: 5′-GTTCGCAGCGTCACCCGGATCTTCG-3′ (SEQ ID NO:4) and Reverse: 5′-AGGCTTGCAGAATGCGGAACACC-3′ (SEQ ID NO:5)). The following conditions were used for PCR amplification of PGK-1: 1 cycle of 95° C. for 3 mins; 33 cycles of 95° C. for 30 s, 65° C. for 30 s, 72° C. for 20 s; 1 cycle of 72° C. 5 mins and A_{2A} receptor: 1 cycle of 95° C. for 3 mins; 31 cycles of 95° C. for 30 s, 62° C. for 30 s, 72° C. for 20 s; 1 cycle of 72° C. 5 min.

Immunoprecipitation of the chromatin complexes formed when HLMVEC were exposed to hypoxia showed significant enrichment of the A_{2A} promoter fragment with the specific HIF-2α antibody when compared to the normoxic control or the mock antibody control (FIG. 3C). Similar enrichment of PGK-1 was also observed in HLMVEC under identical conditions and was used as a positive control (FIG. 3C).

### Example 4: This Example Illustrates that Expression of A_{2A} Receptor is Involved in Promoting Cellular Proliferation

Proliferation of HLMVEC was measured using [³H]-thymidine incorporation. About 20,000 cells were plated in each well of a 24-well plate in endothelial cell complete medium. After 24 h, cells were washed once with HBSS and serum-starved in EBM-2 medium containing 1% FBS, hydrocortisone, ascorbic acid and GA-1000. After 24 h, cells were incubated with [³H] thymidine (1 μCi/well) in the presence or absence of the adenosine A_{2A} receptor agonist, CGS-21680, at varying concentrations for an additional 24 h. Subsequently, cells were washed twice with ice-cold PBS, precipitated with 0.1 N perchloric acid, and solubilized with 0.01 N NaOH containing 0.1% SDS prior to scintillation counting. Similarly, proliferation was also measured in cells transduced with Ad.A_{2A} or Ad.LacZ at a multiplicity of infection of 10 pfu/cell.

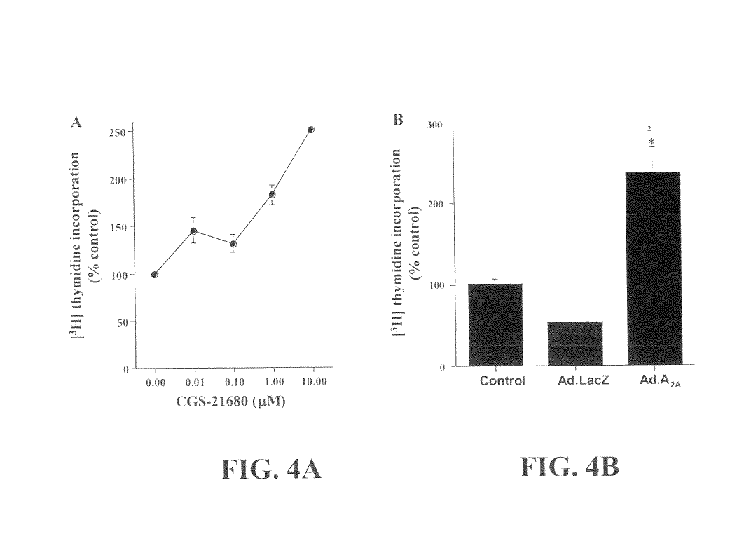

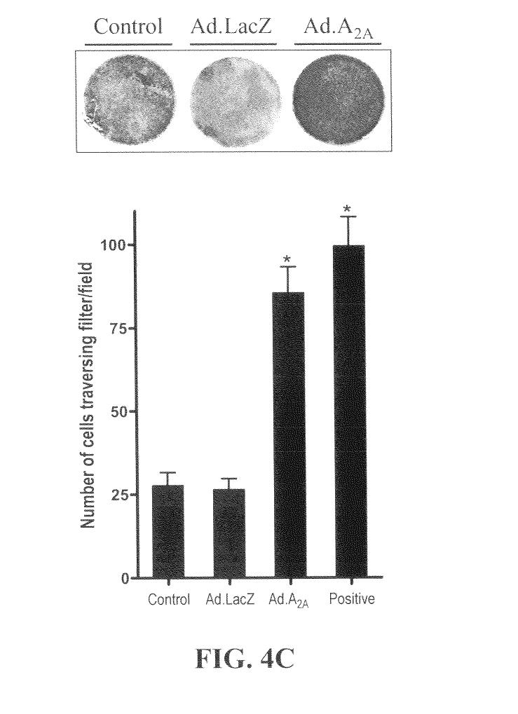

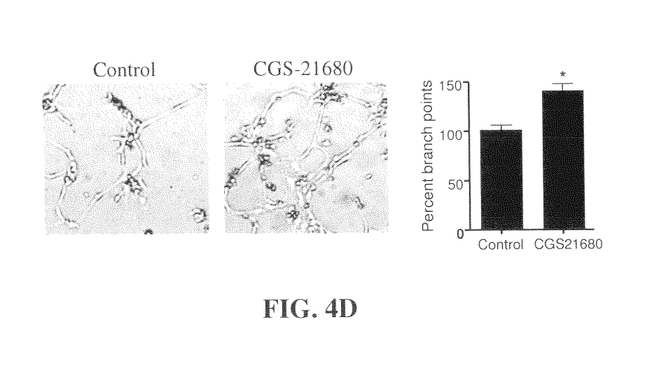

As shown in FIG. 4A, activation of adenosine A_{2A} receptor by the agonist CGS-21680 increased cellular proliferation in a dose-dependent manner. Since hypoxia and HIF-2α increased A_{2A} receptor expression, to investigate whether A_{2A} receptor by itself could alter cellular function, A_{2A} receptor was overexpressed using an adenoviral vector and cellular proliferation was measured as assessed by ³[H]thymidine incorporation. As shown in FIG. 4B, cellular proliferation increased significantly in the presence of overexpressed A_{2A} receptor when compared to control non-transduced cells or the Ad.LacZ-transduced cells.

### Example 5: This Example Illustrates that A_{2A} Receptor is Involved in Promoting Cellular Migration

Since HIF-2α promotes migration of endothelial cells (Tanaka et al., Lab Invest 85, 1292, 2005), it was determined whether adenosine A_{2A} receptor also could increase endothelial cell migration. Angiogenic migration assay was performed as follows. HLMVEC's were either untransduced, transduced with Ad.A_{2A} or with Ad.LacZ at a multiplicity of infection (m.o.i). of 10 pfu/cell. Twenty-four hours after transduction, cells were split and 100,000 cells were plated on a fibronectin-coated insert in EBM-2 medium containing 0.1% FBS, hydrocortisone, ascorbic acid and GA-1000. Prior to plating cells, inserts were coated with 50 μg/ml fibronectin solution in PBS by adding 0.3 ml of the solution to the lower side of the insert and kept at 4° C. for 24 h. Just before adding cells, the inserts were washed twice with PBS to remove unbound fibronectin. Cells were incubated in a humidified cell culture incubator with 5% CO₂, balance air, for an additional 24 h, after which they were washed twice with PBS followed by fixation with 95% EtOH. The inserts were then stained with crystal violet and washed with water to remove unincorporated dye. Stained cells on the apical side of the insert were removed using a swab. The membrane was cut along the edges and scanned for photography. A minimum of eight frames per membrane was collected, and cells in each frame were counted. The mean number of cells per frame was plotted.

As shown in FIG. 4C, migration of HLMVEC across a fibronectin-coated membrane increased in response to increased A_{2A} receptor expression. There was increased migration of cells transduced with Ad.A_{2A} compared to both the Ad.LacZ control and the non-transduced control.

### Example 6: This Example Illustrates that A_{2A} Receptor is Involved in Promoting Cellular Branhing

Angiogenesis in HLMVEC was assessed using the Matrigel tube formation assay. Growth factor-reduced Matrigel matrix was coated onto 12-well plates and allowed to solidify at 37° C. for 30 min. HLMVEC's were then trypsinized and plated onto the Matrigel in the absence of growth factors or serum and incubated at 37° C. in a CO₂ incubator. The A_{2A} receptor agonist, CGS-21680, or the diluent control was included both in the Matrigel matrix and the overlying medium. Four hours after plating of cells, three randomly chosen fields from each well were photographed. Branch points were counted and plotted.

As shown in FIG. 4D, activation of adenosine A_{2A} receptor by the agonist CGS-21680 increased cell sprouting resulting in formation of branches relative to control cells.

### Example 7: This Example Illustrates that HIF-2α, not HIF-1α, Regulates the Expression of the A_{2A} Receptor

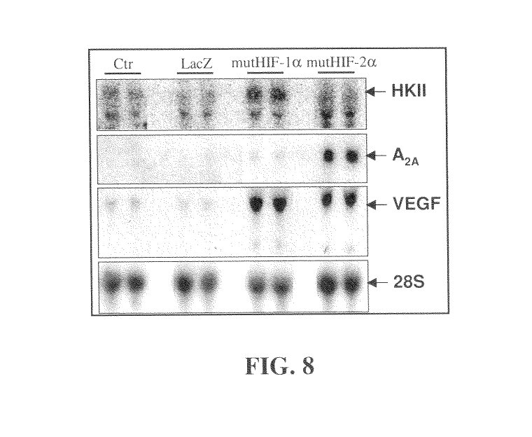

In order to assess whether HIF-1α, HIF-2α or both regulate the expression of adenosine A_{2A} receptor, HLMVEC were adenovirally transduced with mutated HIF-1α and mutated HIF-2α. These HIFs, mutated at critical proline residues, enabled them to function in air (21% O₂), which otherwise would have been degraded under non-hypoxic conditions. Cells were transduced with Ad.LacZ, Ad.mutHIF-1α and Ad.mutHIF-2α at a mulitiplicity of infection of 10 pfu/cell. Twenty four hours post transduction, cells were harvested in GITC and total RNA purified using the CSCl method. After purification, a total of 15 ug of RNA was loaded in each well. After probing for HK-II, the blots were stripped and reproned for A2a, VEGF and 28S in that order. As shown in FIG. 8, only HIF-2α regulate A_{2A} receptor expression. However both HIF-1α and HIF-2α regulate VEGF and only HIF-1α regulates hexokinase-II (HKII).

### Example 8: This Example Illustrates that Adenosine A_{2A} Receptor Activation Promotes Tube Formation

MB114 cells (a microvascular endothelial cell line) were plated on collagen gel in presence of absence of the A_{2A} receptor agonist CGS-21680 at a density of 80000 cells/well using a 24 well plate. After incubation for 5 days, photos from each well were taken randomly. FIG. 8 shows representative photographs showing formation of tubes.

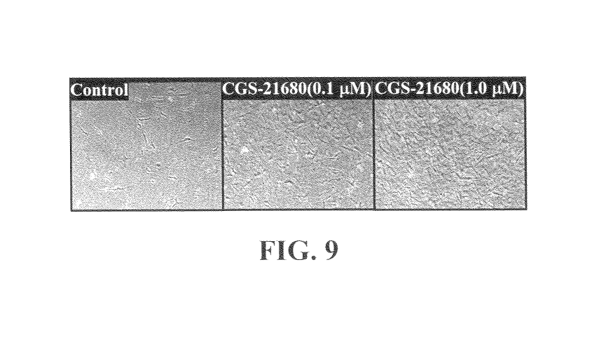

As can be seen from FIG. 9, exposure to the agonist increased tube formation and capillary branching in a dose dependent manner.

### Example 9: This Example Illustrates that Adenosine A_{2A} Receptor is Expressed in Different Tumor Stages of Lung Cancer

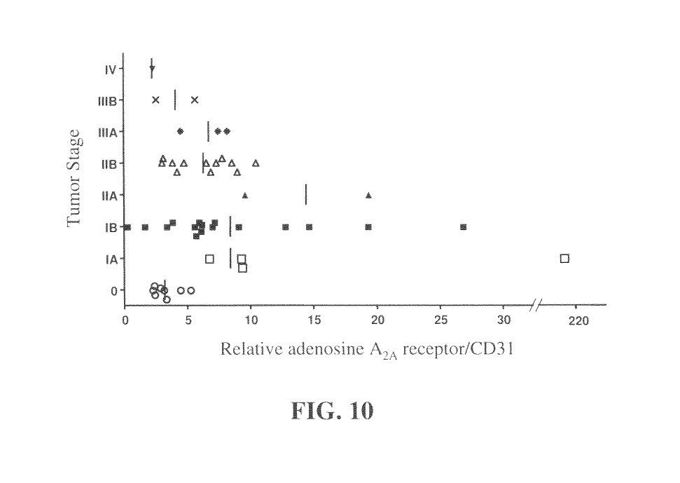

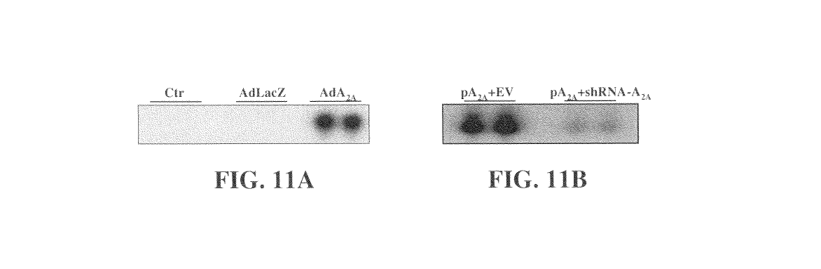

Real time RT-PCR was carried out for A_{2A} receptor and the endothelial marker CD31 using specific primers and probe for each protein. In order to assess endothelial contribution of the receptor, relative fold change of A_{2A} receptor was normalized to expression of CD31. As shown in FIG. 10, there was a marked increase in receptor expression in a number of patient samples representing different tumor stages.

### Example 10: This Example Illustrates the Knockdown Ability of the Adenoviral Shuttle Vector Expressing the siRNA Against the A_{2A} Receptor

An adenoviral shuttle vector expressing A_{2A} receptor (pA_{2A}), as well as an adenoviral vector expressing siRNA against the A_{2A} receptor (siRNA-A_{2A}) were constructed using standard molecular biological techniques. The vectors were expressed in HLMVEC using transient transfection assays. Expression of A_{2A} receptor mRNA was detected using the Northern blot technique as described before.

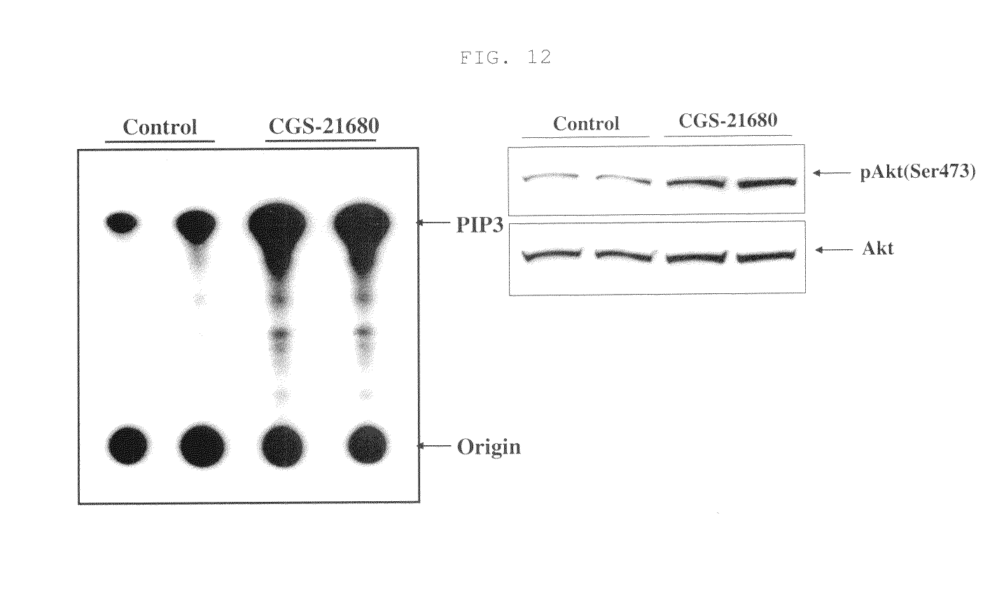

As shown in FIG. 11A, A_{2A} receptor expression was detected in cells transduced with the adenovirus carrying the A_{2A} receptor gene. As shown in FIG. 11 B, A_{2A} receptor expression was detected in cells transfected with adenoviral shuttle vector expressing A_{2A} receptor co-transfected with the empty vector (pA_{2A}+EV). However, the expression of A_{2A} receptor was knocked out when the pA_{2A} was cotransfected with the shuttle vector expressing siRNA against A_{2A} (pA_{2A}+shRNA-A_{2A}). The results in FIG. 11B are shown in duplicate.

### Example 11: This Example Illustrates that Activation of the A_{2A} Receptor Increases PI 3-Kinase Activity

HLMVECs were cultured on 100 mm dishes. Cells were serum starved for 24 h before treating with 1 μM of CGS-21680 (Adenosine A_{2A} receptor agonist) or the diluent control. Following treatment, lysates were prepared and −500 μg of protein was incubated with 20 μl of anti-p85-conjugated agarose (200 μg anti p85/200 μl agarose) for 2 h, after which the complex was precipitated. PI3-kinase activity was measured in the immunoprecipitate.

FIG. 12 shows a representative autoradiogram demonstrating PI 3-kinase-mediated phosphorylation of phosphoinositides, PIP3 and expression of phosphorylated Akt (a downstream target of PI 3-Kinase) measured by Western blotting (right panel). As can be seen in FIG. 12, activation of the A_{2A} receptor increased PI3-kinase activity.

### Example 12: This Example Illustrates the Pattern of A_{2A} and A_{2B} Receptor Expression in Maturing Baboon Lung

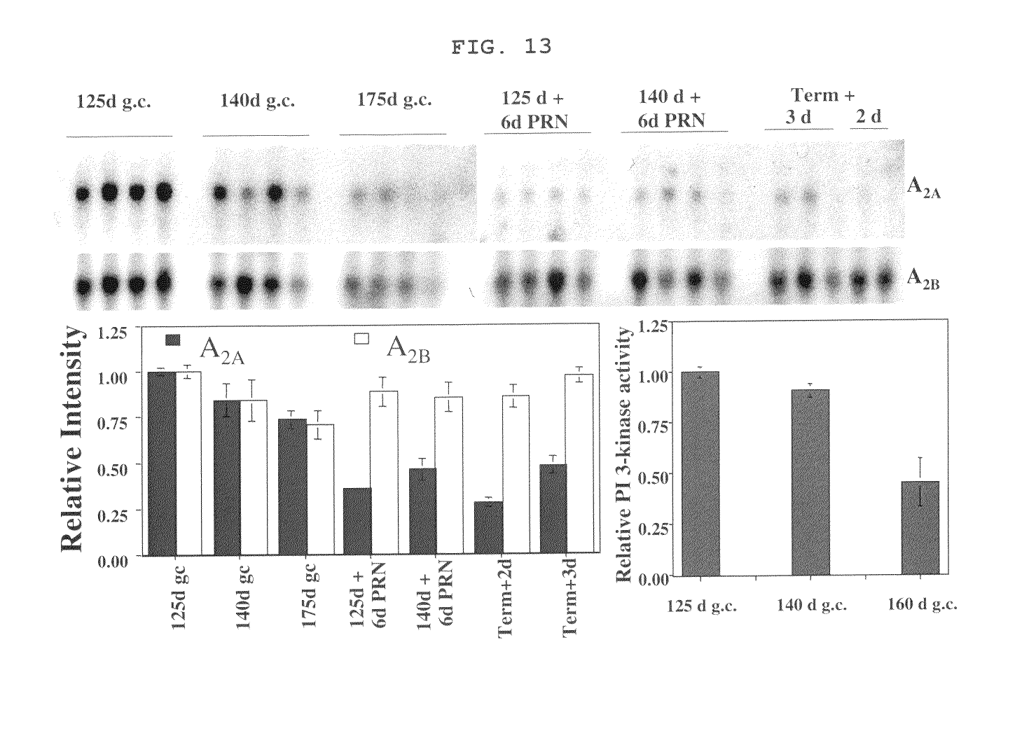

Frozen lung tissues from gestational control (GC), Gestational control born prematurely and provided oxygen as needed (PRN; latin “pro re nata” meaning as needed) and Term baboons were obtained and harvested in guanidine isothiocyanate solution. Total cell RNA was then purified with CsCl centrifugation. Equal amounts of RNA (15 μg) were resolved on a 1% agarose-2.5 M formamide gel in a 20 mM MOPS buffer, pH 7.4, containing 1 mM EDTA. A standard Northern blot procedure was used to transfer the RNA to a nylon membrane. Blots were hybridized with the A2a and A2b probe and autoradiographed. The top panel of FIG. 13 is a representative blot and lower left figure shows the quantification of relative intensity with 28S RNA as control. Protein lysates were also obtained from the frozen tissue and analysed for PI3K activity (lower right corner) as described in Example 11.

As shown in FIG. 13, A_{2A} receptor expression is higher in the lung undergoing development and decreases as the lung nears full development.

The foregoing description of the present invention has been presented for purposes of illustration. The description is not intended to limit the invention to the form disclosed herein. Consequently, variations and modifications commensurate with the above teachings, and the skill or knowledge of the relevant art, are within the scope of the present invention. The embodiments described hereinabove are further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

Each publication and reference cited herein is incorporated herein by reference in its entirety.

1. A method to diagnose and treat a patient with a cancer that is associated with HIF-2α expression, comprising:
a) detecting the expression of adenosine A_{2A} receptor (A_{2A}) in a sample of tumor cells from a patient;
b) comparing the level of expression of A_{2A} detected in the patient sample to a level of expression of A_{2A} in a non-tumor cell control sample;
c) diagnosing the patient as having a cancer that is associated with HIF-2α, if the expression level of A_{2A} in the patient's tumor cells is statistically higher than the expression level of A_{2A} in the non-tumor cell control; and
d) treating the patient diagnosed in step (c) by administering to the patient an siRNA against the A_{2A} receptor.

2.-5. (canceled)

6. The method of claim 1, wherein expression of A_{2A} is detected by measuring amounts of transcripts of the gene in the tumor cells.

7. The method of claim 1, wherein expression of A_{2A} is detected by detecting the A_{2A} protein.

8. The method of claim 1, wherein the non-tumor cell control is a cell of the same type as the tumor cell.

9. The method of claim 1, wherein the non-tumor cell control is an autologous, non-cancerous cell from the patient.

10. The method of claim 1, wherein control expression levels of A_{2A} have been predetermined.

11.-20. (canceled)

21. A method to treat a patient having a cancer that is associated with HIF-2α expression comprising administering to the patient an siRNA against the A_{2A} receptor.
